(19) United States
(12) Patent Application Publication
(10) Pub. No.: US 2017/0027772 A1
(43) Pub. Date: Feb. 2, 2017

(54) Feminine Hygiene Article Holding Apparatus

(71) Applicant: Valena C. Agee, Montgomery, AL (US)

(72) Inventor: Valena C. Agee, Montgomery, AL (US)

(21) Appl. No.: 14/810,591

(22) Filed: Jul. 28, 2015

(51) Int. Cl. A61F 13/551; B65D 25/14

(52) U.S. Cl. CPC A61F 13/5515; A61F 13/5518; B65D 25/14

(57) ABSTRACT

A feminine hygiene article holding apparatus includes a container that has a bottom wall and a perimeter wall is attached to and extends upwardly from the bottom wall. The container has a height less than six inches and includes an outer layer and an inner layer. The inner layer is colored red to indicate the container may contain a hazardous material. A feminine hygiene product may be positioned in the container.

### BACKGROUND OF THE DISCLOSURE

### Field of the Disclosure

The disclosure relates to feminine hygiene article holding devices and more particularly pertains to a new feminine hygiene article holding device for holding a use feminine hygiene absorbent member until it can be properly disposed of.

### SUMMARY OF THE DISCLOSURE

An embodiment of the disclosure meets the needs presented above by generally comprising a container that has a bottom wall and a perimeter wall is attached to and extends upwardly from the bottom wall. The container has a height less than six inches and includes an outer layer and an inner layer. The inner layer is colored red to indicate the container may contain a hazardous material. A feminine hygiene product may be positioned in the container.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of a feminine hygiene article holding apparatus according to an embodiment of the disclosure.

FIG. 2 is a front view of an embodiment of the disclosure.

FIG. 3 is a side view of an embodiment of the disclosure.

FIG. 4 is a cross-sectional view of an embodiment of the disclosure taken along line 4-4 of FIG. 3.

### DESCRIPTION OF THE PREFERRED EMBODIMENT

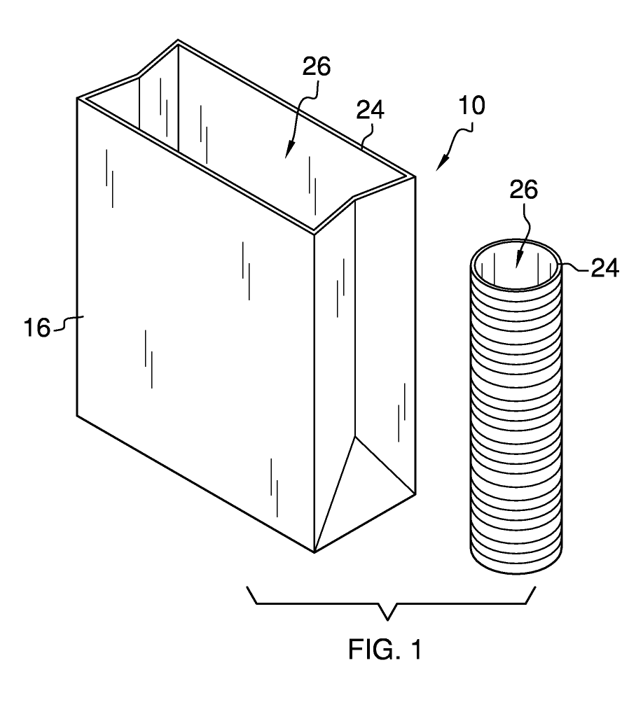

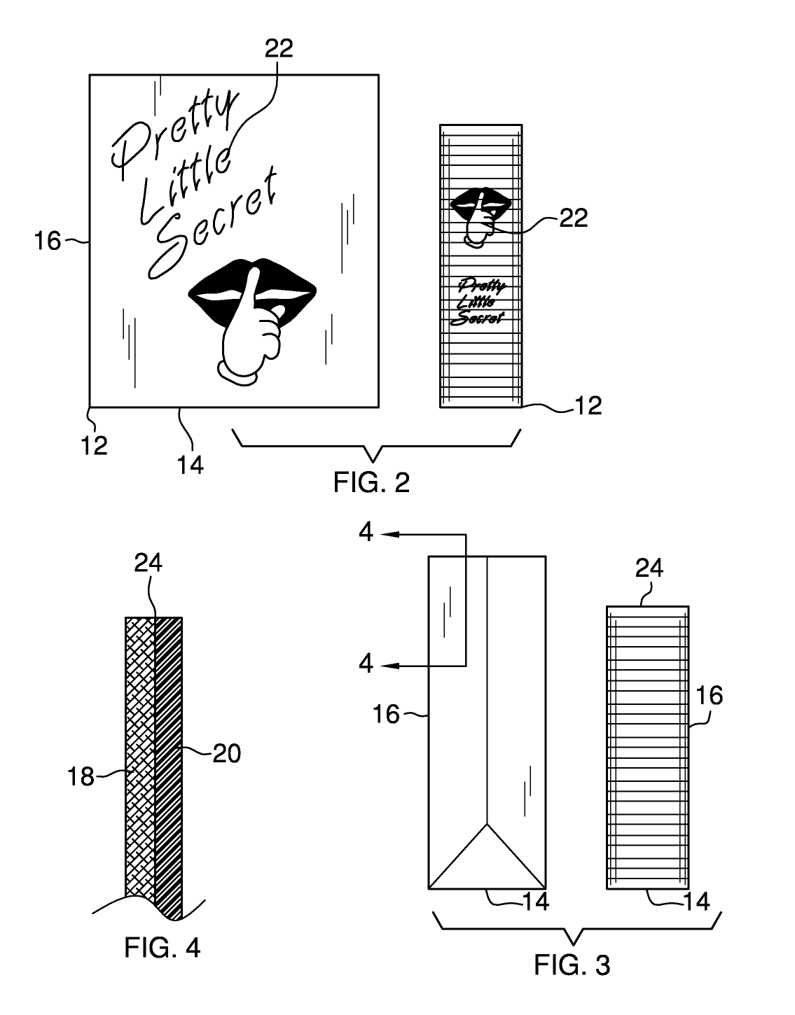

With reference now to the drawings, and in particular to FIGS. 1 through 4 thereof, a new feminine hygiene article holding device embodying the principles and concepts of an embodiment of the disclosure and generally designated by the reference numeral 10 will be described.

As best illustrated in FIGS. 1 through 4, the feminine hygiene article holding apparatus 10 generally comprises a container 12 that has a bottom wall 14 and a perimeter wall 16, which is attached to and extends upwardly from the bottom wall 14. The perimeter wall 16 has an upper edge 24 defining an opening 26 into the container 12. The container 12 has a height less than six inches. The container includes 12 an outer layer 18 and an inner layer 20. The inner layer 20 is colored red to indicate the container may contain a hazardous material. The inner layer 20 is fluid impervious. The container 12 may be made out of a flexible material.

As shown in the Figures, the container 12 may either be rectangular shaped or cylindrically shaped. For the rectangular shaped container 30, the bottom wall 14 has a rectangular shape has a length and width each less than four inches. For the cylindrically shaped container 32, the bottom wall 14 is circular shaped and has a diameter less than 3 inches.

In use, a feminine hygiene product such as a tampon or feminine napkin, which has been used, is positioned in the container 12. The container 12 may then be stored within a purse or other bag until it can be properly disposed. The red inner layer 20 will signal to others not to access the contents of container 12. The outer layer 18 may have decorative indicia 22 thereon as shown in the figures to make the container 12 more aesthetically pleasing and therefore more likely to be used.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure.

I claim:

1. A holding apparatus configured to hold and temporarily store a used absorbent material feminine hygiene product, said apparatus comprising:
a container having a bottom wall and a perimeter wall being attached to and extending upwardly from said bottom wall, said container having a height less than six inches, said container including an outer layer and an inner layer, said inner layer being colored red to indicate said container may contain a hazardous material; and
wherein a feminine hygiene product may be positioned in said container.

2. The apparatus according to claim 1, wherein said inner layer is fluid impervious.

3. The apparatus according to claim 1, wherein said bottom wall has a rectangular shape and having a length and width each less than four inches.

4. The apparatus according to claim 1, wherein said bottom is circular such that said container is cylindrically shaped.

5. The apparatus according to claim 4, wherein said bottom has a diameter less than three inches.

6. The apparatus according to claim 5, wherein said inner layer is fluid impervious.

7. A holding apparatus configured to hold and temporarily store a used absorbent material feminine hygiene product, said apparatus comprising:
a container having a bottom wall and a perimeter wall being attached to and extending upwardly from said bottom wall, said container having a height less than six inches, said container including an outer layer and an inner layer, said inner layer being colored red to indicate said container may contain a hazardous material, said inner layer being fluid impervious;
said bottom wall having a rectangular shape and having a length and width each less than four inches; and
wherein a feminine hygiene product may be positioned in said container.
